(19) United States
(12) Patent Application Publication
(10) Pub. No.: US 2010/0256676 A1
(43) Pub. Date: Oct. 7, 2010

(54) SUTURE CLIPS FOR SECURING SUTURES

(76) Inventors: Alan R. HAY, Salem, OR (US); Greg A. Shaver, Springfield, OR (US)

Correspondence Address: Mohr Intellectual Property Law Solutions, P.C., 16814 NW Oak Creek Dr., Beaverton, OR, 97006, US

(21) Appl. No.: 12/419,104

(22) Filed: Apr. 6, 2009

(51) Int. Cl. A61B 17/04

(52) U.S. Cl. 606/232

(57) ABSTRACT

Suture clips include a first clip portion and a second clip portion complimentarily configured with the first clip portion for securing a suture between the first and second clip portions when the first and second clip portions are selectively pressed together. The first and second clip portions include base members and clamp portions, at least one of the clamp portions defining a directional locking region configured to permit the suture to move in only one direction.

### BACKGROUND

Many different surgical techniques and medical procedures involve cutting or puncturing tissue. Sutures are used to sew tissue back together to close any such openings, cuts, or incisions. Sutures are also used to join together different tissue portions and to apply pressure to tissue. For a suture to effectively sew, join, or apply pressure to tissue on an ongoing basis, the suture must be secured in a desired position.

Sutures may be secured in a variety of ways. Surgeons sometimes tie a suture with knots, but this does not always satisfactorily secure the suture and can be difficult to do in certain applications, such as laparoscopic surgery. Surgeons sometimes use anchors to secure sutures. In other instances, surgeons utilize a suture clip that selectively fastens the suture to then serve as an anchor.

Existing suture clips do not fully solve the problems incumbent with securing a suture during surgery. For example, known suture clips do not allow the surgeon to conveniently and precisely adjust the pressure applied by a suture onto an organ or tissue. Further, known suture clips do not adequately distribute the pressure applied by the suture and suture clip over a sufficiently large area of the organ or tissue to reduce the pressure to acceptable levels. Excessive pressure applied to tissue can damage the tissue and prolong recovery time. Moreover, known suture clips do not provide fully convenient and effective means to selectively remove the suture clip. Another way known suture clips do not fully address the needs of doctors and patients is that they do not incorporate absorbent materials that can reduce bleeding and increase recovery time.

Examples of suture clips are disclosed in the following U.S. patent and patent application references, which are hereby incorporated by reference for all purposes: U.S. Pat. Nos. 3,976,079, 4,291,698, 4,387,489, 4,620,541, 5,078,731, 5,160,339, 5,234,449, 5,330,442, 5,409,499, 5,474,572, 5,951,590, 6,254,615, 7,001,412, 7,108,710, and 2005/0004602.

### SUMMARY

The present disclosure is directed to suture clips. Suture clips include a first clip portion and a second clip portion complimentarily configured with the first clip portion for securing a suture between the first and second clip portions when the first and second clip portions are selectively pressed together. The first and second clip portions include base members and clamp portions, at least one of the clamp portions defining a directional locking region configured to permit the suture to move in only one direction.

### BRIEF DESCRIPTION OF THE DRAWINGS

FIG. 1 is a perspective view of an organ with two suture passing through it, each suture being secured on at a top end of the organ by a first example of a suture clip.

FIG. 2 is a perspective view of a second example of a suture clip, which is securing free ends of sutures used to stitch together portions of an organ.

FIG. 3 is a plan view of the suture clip shown in FIG. 2 securing free ends of sutures from an anchor position of a first organ portion.

FIG. 4 is a perspective view of the first example of a suture clip in an open position.

FIG. 5 is a side elevation cross section view of directional locking regions of the suture clip of FIG. 4 taken about line 5-5 in FIG. 4.

FIG. 6 is a plan view of an example of a bottom of the suture clip of FIG. 4.

FIG. 7 is a side elevation view of the suture clip of FIG. 4 showing a hemostatic material on a pressure face.

FIG. 8 is a perspective view of an example of a first clamp portion of the suture clip of FIG. 4 including a directional locking region with horizontal ridges.

FIG. 9 is a perspective view of an example of a first clamp portion of the suture clip of FIG. 4 including a directional locking region with a combination of horizontal and angled ridges.

FIG. 10 is a perspective view of an example of a first clamp portion of the suture clip of FIG. 4 including a directional locking region with a plurality of tilted bristles.

FIG. 11 is a perspective view of an example of a first clamp portion of the suture clip of FIG. 4 including a directional attachment region with a plurality of overlapping plates.

FIG. 12 is a perspective view of the second example of a suture clip in a closed position with two sutures secured by the suture clip.

FIG. 13 is a top plan view of the suture clip of FIG. 12 shown pivoting between open and closed positions.

FIG. 14 is a perspective view of an example of the suture clip of FIG. 12 including buttresses extending from first and second clamp portions.

FIG. 15 is a perspective view of an example of the suture clip of FIG. 12 that translates between and open position and a closed position.

FIG. 16 is a perspective view of an example of the suture clip of FIG. 12 that includes buttresses and a latch with a notch.

FIG. 17 is a perspective view of an example of the suture clip of FIG. 12 that includes buttresses and first and second contact faces that pivot between tilted and upright positions.

FIG. 18 is a perspective view of an example of the suture clip of FIG. 4 with a unitary base member and clamp portions that pivot.

FIG. 19 is a perspective view of a third example of a suture clip, which includes cleats for securing a suture.

FIG. 20 is a perspective view of another example of the suture clip shown in FIG. 19, the suture clip including arms for securing a suture.

### DETAILED DESCRIPTION

The disclosed suture clips will become better understood through review of the following detailed description in conjunction with the drawings and the claims. The detailed description, drawings, and claims provide merely examples of the various inventions described herein. Those skilled in the art will understand that the disclosed examples may be varied, modified, and altered without departing from the scope of the inventions as defined in the claims, and all equivalents to which they are entitled. Many variations are contemplated for different applications and design considerations; however, for the sake of brevity, each and every contemplated variation is not individually described in the following detailed description.

Throughout the following detailed description, a variety of suture clip examples are provided. Related reference numbers (e.g., 12, 112, 212) will be used for related features in each example. Related features may be identical, similar, or dissimilar in different examples. For the sake of brevity, related features will not be redundantly explained in each example. Instead, the use of related numbers will cue the reader that the feature with a related number may be similar to the related feature in an example explained previously. Features specific to a given example will be described in that particular example. The reader should understand that a given feature need not be the same or similar to the specific portrayal of a related feature in any given figure or example.

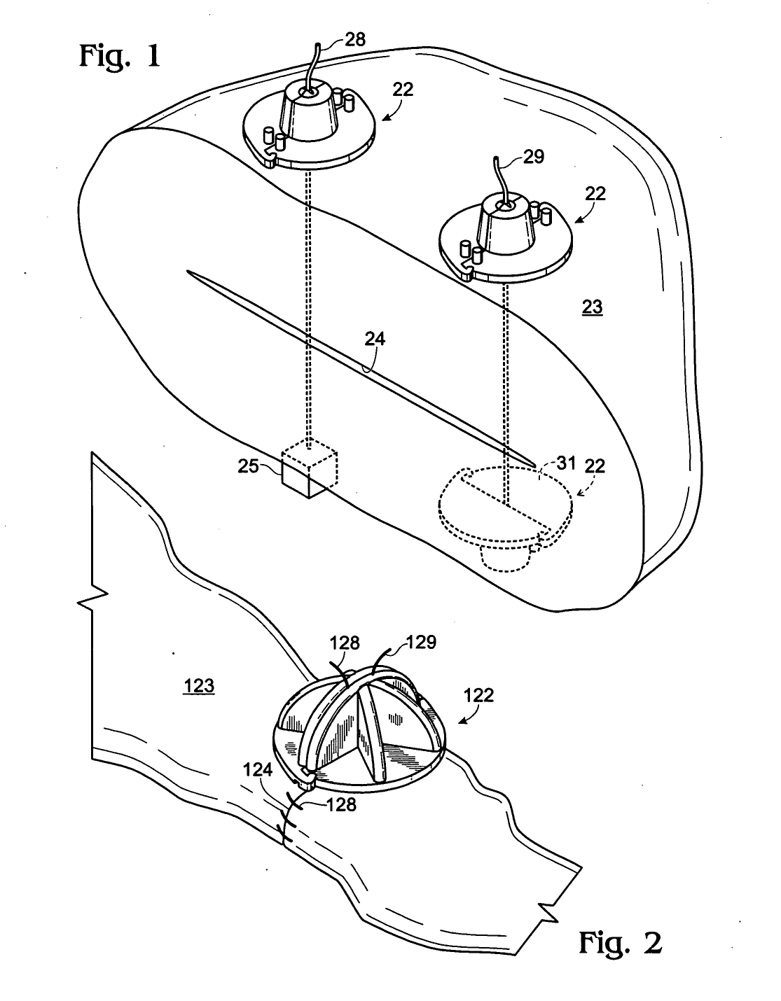

Three first suture clips 22 are shown in FIG. 1 securing first and second sutures 28 and 29 extending through an organ 23 having a cleft or incision 24. Suture 28 is secured by first suture clip 22 (or simply clip 22) on a top side of organ 23 and by an anchor 25 on a bottom side of organ 23 (note, all relative references, such as top, bottom, left or right are based on the orientations depicted in the relevant figures being described). Anchor 25 may be made from a material designed to dissolve over time, such as Lapra-Ty® by Ethicon/Johnson and Johnson. Suture 29 is secured by clips 22 on both the top and bottom of organ 23. Sutures 28, 29 may be any suture commonly employed during medical surgery, such as Vicryl® brand sutures made by Ethicon.

Clip 22 and suture 28 cooperatively facilitate applying pressure to organ 23. Tension developed in suture 28 imparts pressure to the surface of organ 23 via clip 22. As will be explained in more detail below, clip 22 allows suture 28 to move relative to clip 22 in a direction towards the top of the page in FIG. 1 and restricts suture 28 from moving relative to clip 22 in a direction towards the bottom of the page. The same selective relative motion applies between second suture 29 and clips 22 securing second suture 29. However, the orientation of clip 22 securing suture 29 on the bottom of organ 29 in FIG. 1 dictates that suture 29 is allowed to move toward the bottom of the page and restricted from moving toward the top of the page.

An example of a second suture clip 122 securing first and second sutures 128 and 129 is shown in FIG. 2. In FIG. 2, sutures 128, 129 have sewn together an incision 124 in an organ 123. Free ends of sutures 128, 129 are secured by clip 122. Clip 122 can be cinched tighter upon organ 123 and enable incision 124 to be sewn together more tightly. Clip 122 can be used to apply pressure to organ 123 and to tightly sew together incision 124 because it allows sutures 128, 129 to move towards the top of the page in FIG. 2, but restricts the sutures from moving toward the bottom of the page.

FIG. 3 depicts clip 122 being utilized in another application. In FIG. 3, clip 122 is securing sutures 128, 129 and serving as an anchor. FIG. 3 shows clip 122 joining together first and second portions (shown on right and left sides of FIG. 3, respectively) of a tubular structure 123, such as a ureter, at incision faces 124.

As shown in FIG. 3, sutures 128, 129 are secured by clip 122 on an external side of the first portion of organ 123. First and second needles 184, 185 are coupled to the leading ends of sutures 128, 129 and enable the sutures to extend through a wall of the first portion of organ 123. Needles 184, 185 are also used to extend sutures 128, 129 through a wall of the second portion of organ 123.

When tension is applied to sutures 128, 129, clip 122 is pulled toward the second portion of organ 123. Because clip 122 is located on an external side of the first portion of organ 123, clip 122 pushes the first portion of organ 123 into contact with the second portion as clip 122 is pulled toward the second portion. Securing the leading ends of sutures 128, 129, such as with another clip 122, a clip 22, or by some other means, secures the first and second portions of organ 123 together.

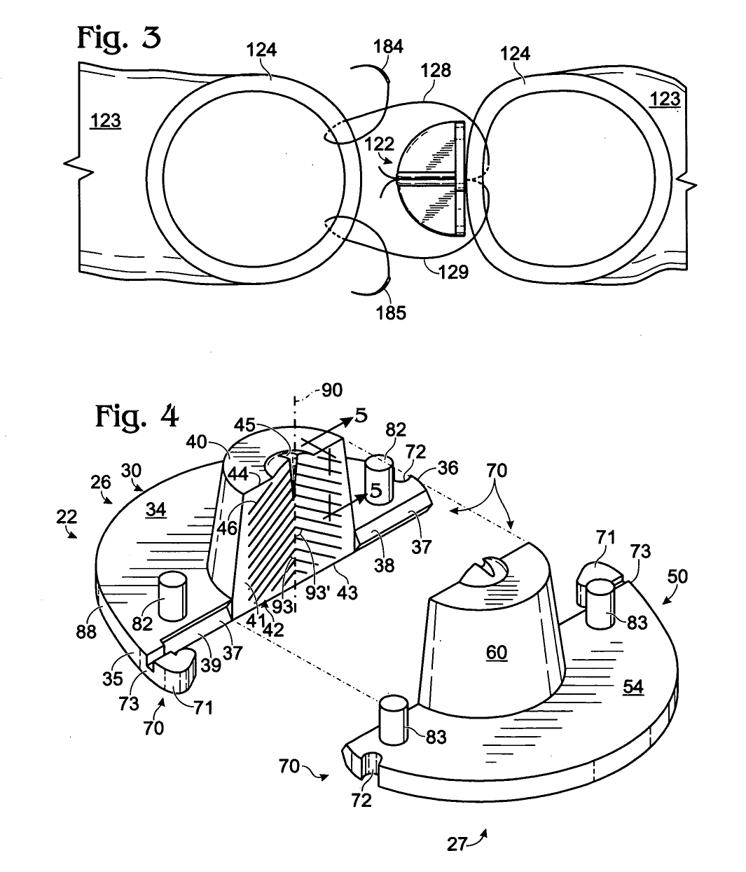

Turning attention to FIG. 4, clip 22 is described in more detail. Clip 22 includes a first clip portion 26 and a second clip portion 27 that are cooperatively configured to secure a suture between them. In the example shown in FIG. 4, first clip portion 26 is identical to second clip portion 27. However, in other examples, first clip portion 26 is structurally different than second clip portion 27.

Clip portion 26 includes a first base member 30, a first clamp portion 40, and first posts 82. Clip portion 27 includes a second base member 50, a second clamp portion 60, and second posts 83. As first and second clip portions 26, 27 are identical in the example shown in FIG. 4, each of first and second base members 30, 50, first and second clamp portions 40, 60, and first and second posts 82, 83 are identical in the example shown in FIG. 4. However, in other examples, one or more of first and second base members 30, 50, first and second clamp portions 40, 60, and first and second posts 82, 83 are structurally different. For the sake of brevity, first base member 30, first clamp portion 40, and first posts 82 will be described below with the understanding that the description applies equally to second base member 50, second clamp portion 60, and second posts 83, unless noted otherwise.

With reference to FIGS. 1, 4, 6, and 7, base member 30 includes a first pressure face 31, a first support face 34, a first abutment surface 37, and a first rear boundary surface 88. Support face 34 is opposite pressure face 31. Abutment surface 37 extends between pressure face 31 and support face 34 from a first shoulder 35 to a second shoulder 36. Rear boundary surface 88 extends opposite abutment surface 37 between pressure face 31 and support face 34 from first shoulder 35 and second shoulder 36. Structure on second clip portion 27 corresponding to first base member 30, first pressure face 31, first support face 34, first abutment surface 37, and first rear surface 88 is second base member 50, second pressure face 51, second support face 54, second abutment surface 57, and second rear surface 89.

In the example of base member 30 shown in FIGS. 1, 4, 6, and 7, pressure face 31, support face 34, abutment surface 37, and a rear boundary surface 88 collectively define a curved and planar disk. In other examples, the disk is rectangular, square, triangular, oval, circular or an irregular shape. Further, in some examples the disk is curved or rounded instead of planar, such as to more accurately conform with an opposing curved surface of an organ. The disk serves to support the components of clip 22 on first support face 34. Further, the disk distributes the weight and tension forces existing in clip 22, as well as in sutures retained by clip 22, over a relatively large surface area of organ 23 corresponding to the area of pressure face 31.

As shown in FIG. 1, pressure face 31 is configured to face and be in contact with organ 23. Pressure face 31 is designed to have a relatively large surface area to reduce the pressure exerted onto organ 23 by clip 22, which bears the tension developed in a suture retained by clip 22. Pressure face 31 may have an area of between 1 square millimeters and 1300 square millimeters, as suited to a given application.

For example, when clip 22 is intended for use during laparoscopic surgery, pressure face 31 may have an area of between 20 and 180 square millimeters to optimize the ability of clip 22 to insert into the body of a patient through a relatively small opening while still being able to effectively distribute pressure over organ 23. In examples where clip 22 is used in microsurgical procedures, the area of the pressure face could be minimized to the extent allowed by current and future manufacturing techniques, such as plastic molding techniques. In “open” surgery applications, the size of the pressure face generally need not be limited by the size of the entry point into the body of a patient. Thus, pressure faces having larger areas, such as 314 square millimeters or more, may be used.

In the example shown in FIG. 6, pressure face 31 includes a plurality of first texture elements 32. Texture elements 32 increase the coefficient of friction of pressure face 31, which functions to reduce incidence of pressure face 32 and clip 22 sliding relative to organ 23. Clip 22 sliding relative to organ 23 can irritate organ 23 and inhibit the ability of organ 23 to heal after surgery. In the example shown in FIG. 6, second pressure face 51 includes a plurality of second texture elements 52. Texture elements 52 may be the same or different than texture elements 32. In some examples, neither pressure face 31 nor pressure face 51 include texture elements.

In the example shown in FIG. 7, a coating layer 33 is applied to pressure face 31. Coating layer 33 may include a material having active ingredients with pharmacological properties, including anti-inflammatory properties, anti-bacterial properties, or coagulation inhibiting or promoting properties. In some examples, coating layer 33 includes a hemostat material, such as Gelfoam® brand hemostats made by Pfizer or Surgicel® brand hemostats made by Ethicon.

Abutment surface 37 is configured to cooperatively abut with abutment surface 57, such as shown in FIG. 7. In the example shown in FIG. 4, abutment surface 37 defines a projection 38 and a recess 39 to stabilize clip 22 in a direction corresponding to an imaginary line 90 when first and second clip portions 27, 28 are selectively pressed together. Projection 38 is complimentarily configured with a recess defined in second abutment surface 57, which is identical to recess 39 defined in first abutment surface 37. Likewise, recess 39 is complimentarily configured with a projection defined in second abutment surface 57, which is identical to projection 38. In some examples, first and second abutment surfaces 37, 57 do not include projections or recesses, but instead present substantially flat faces.

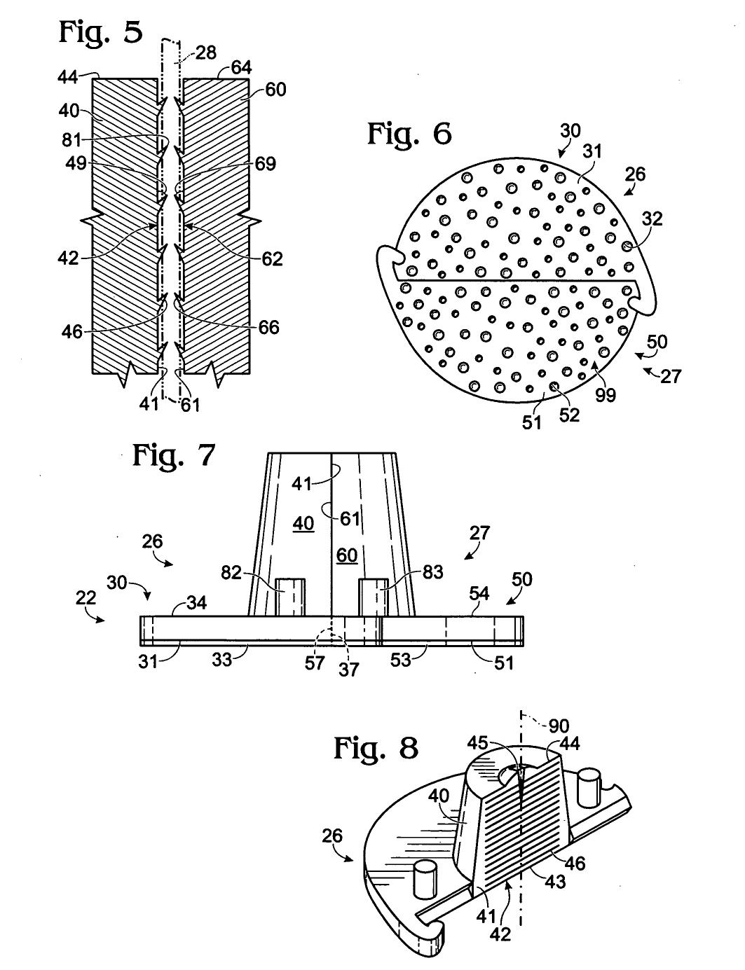

As shown in FIG. 5, clamp portion 40 cooperates with clamp portion 60 to secure suture 28 between them when first and second clip portions 27, 28 are selectively pressed together. Clamp portion 40 includes a first clamp face 41, which defines a first directional locking region 42 and a guide 45. In the example shown in FIGS. 4 and 8-11, guide 45 is a channel defined at or near the center of the width of clamp face 41. The channel has a width slightly larger than the radial width, i.e., diameter of suture 28 to enable a user to insert suture 28 at least partially within the channel to thereby align suture 28 with the center of the width of clamp face 41.

Region 42 includes a first end 43 proximate base member 30 and a second end 44 distal base member 30. Thus, in FIGS. 4 and 8-11, first end 43 is oriented closer to the bottom of the page than second end 44, which is oriented closer to the top of the page.

Region 42 functions to restrict the suture from moving in one direction while allowing the suture to move in an opposite direction. Specifically, in the examples shown in FIGS. 4, 5 and 8-11, region 42 permits suture 28 to move from first end 43 to second end 44 and restricts suture 28 from moving from second end 44 to first end 43. In this manner, region 42 locks suture 28 in place with regard to a selected direction of motion.

Region 42 may include or define a variety of features that facilitate restricting suture 28 from moving in a particular direction. In the example shown in FIGS. 4, 5, 8 and 9, region 42 defines a plurality of ridges 46. In the example shown in FIG. 10, region 42 includes a plurality of bristles 47. In the example shown in FIG. 11, region 42 includes a plurality of overlapping plates 48.

As shown in FIGS. 4, 8 and 9, region 42 may define a variety of ridge patterns. First imaginary line 90 is shown in FIGS. 4, 8 and 9 to serve as a reference aid. Line 90 extends between first end 43 and second end 44. Suture 28 will typically be oriented substantially along or approximately parallel to line 90 when retained between first and second clamp portions 40, 60.

In each of the ridge pattern examples shown in FIGS. 4, 8 and 9, ridges 46 on the left side of line 90 form a mirror image of ridges 46 on the right side of line 90. In other examples, ridges 46 do not form a mirror image across line 90, but instead have different patterns on each side of line 90.

A further common feature of the ridge pattern examples shown in FIGS. 4, 8 and 9 is that ridges 46 extend across region 42 transverse to line 90. As suture 28 will typically be oriented substantially in line with line 90, ridges 46 will typically be transverse to suture 28. Suture 28 being oriented transverse to ridges 46 results in a plurality of ridges acting on suture 28 at different points along its length, which facilitates restricting suture 28 from moving in a given direction.

In the example shown in FIG. 8, ridges 46 extend across region 42 in a series of parallel rows that are substantially perpendicular to line 90. Any variety of spacing between the rows may be selected. In the example shown in FIG. 4, ridges 46 on the left side of line 90 are arranged in a plurality of parallel rows oriented at a first oblique slant angle 93 relative to line 90. On the right side of line 90 in FIG. 4, ridges 46 are arranged in a plurality of parallel rows oriented at a second oblique slant angle 93′ relative to line 90. In the example shown in FIG. 4, first and second oblique slant angles 93, 93′ are equal in magnitude and in other examples they have different magnitudes.

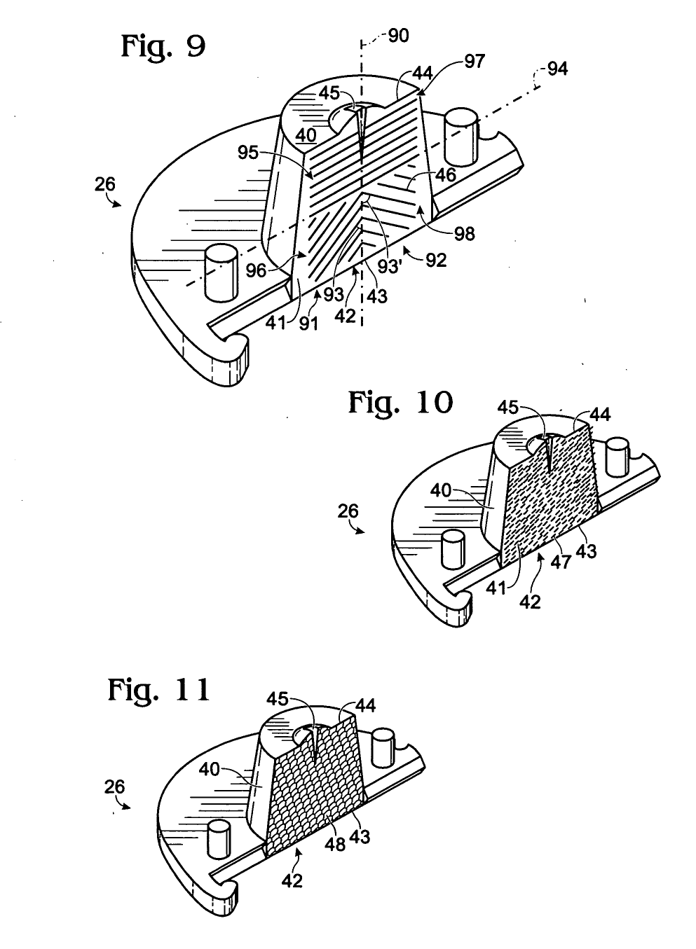

A second imaginary line 94 is provided in FIG. 9 as a further reference aid. Line 94 extends across region 42 and is perpendicular to line 90. Line 90 divides region 42 into a first portion 91 to the left of imaginary line 90 and a second portion 92 to the right of imaginary line 90. Line 94 divides first portion 91 into a subpart A above line 94 and a subpart B below line 94. Moreover, line 94 divides second portion 92 into a subpart C above line 94 and a subpart D below line 94.

In the example shown in FIG. 9, ridges 46 extend across subpart A 95 and subpart C 97 in a series of parallel rows that are substantially perpendicular to line 90. Below line 94 in subpart B 96, ridges 46 are arranged in a plurality of parallel rows oriented at first oblique slant angle 93 relative to line 90. In subpart D 98, ridges 46 are arranged in a plurality of parallel rows oriented at second oblique slant angle 93′ relative to line 90. In the example shown in FIG. 9, first and second oblique slant angles 93, 93′ are equal in magnitude and in other examples they have different magnitudes.

As shown in FIG. 5, ridges 46 are tilted relative to clamp face 41 toward second end 44 at a tilt angle 49. Tilt angle 49 may be any angle between 0 and 180°, and tilt angle 49 is shown in FIG. 5 to be approximately 45° for clarity. Tilt angles of approximately 5-85° are generally effective for restricting suture 28 from moving downward in the orientation shown in FIG. 5 while permitting suture 28 to move upwards. Tilt angles 49 of approximately 95-175° are effective for restricting suture 28 from moving upward in the orientation of FIG. 5 while permitting suture 28 to move downwards.

Ridges 46 in FIG. 8 define relatively sharp edges 81. Edges 81 being relatively sharp facilitate ridges 46 pressing or “biting” into suture 28 and thus restricting suture 28 from moving opposite the direction ridges 46 are pointing. In some examples, edges 81 are less sharp, rounded, or blunt, such as in applications where the user desires a clip that bites less significantly into suture 28.

In some examples, (not pictured) the ridges define a bent hook or claw that terminates in a relatively sharp edge. The hook or claw bends towards the direction that the suture is permitted to move towards. As in the example shown in FIG. 7, the relatively sharp edges of the hook or claw facilitates the ridges biting into a suture.

In the example of clip portion 26 shown in FIG. 10, clamp portion 40 defines a region 42 including a plurality of bristles 47. Bristles 47 are tilted relative to clamp face 41 upwards toward second end 44. The tilt of bristles 47 functions to restrict suture 28 from moving from second end 44 toward first end 43 and to permit suture 28 to move from first end 43 toward second end 44. Bristles 47 may be defined by region 42, such as by a molding process, or may be attached to region 42. Bristles 47 are sufficiently rigid to enable them to collectively resist an operational range of tension forces existing in suture 28.

In the example of clip portion 26 shown in FIG. 11, clamp portion 40 defines a region 42 including a plurality of overlapping plates 48. In FIG. 11, plates 48 form a pattern having an appearance similar to fish scales. In the pattern shown in FIG. 11, a portion of a given plate proximate second end 44 is exposed and a portion of the given plate distal second end 44 is covered by an adjacent plate in the pattern. This pattern enables the exposed portion of each plate to bite into suture 28 when it is pulled toward first end 43, thereby restricting suture 28 from moving toward first end 43. When suture 28 is pulled toward second end 44, the plates do not bite into suture 28 because the edges of the plates proximate first end 43 are covered by an adjacent plate in the pattern. Thus, suture 28 is permitted to move toward second end 44.

Also shown in FIG. 5 is a second directional locking region 62 defined by second clamp face 61 of second clamp portion 60. In the example shown in FIG. 5, region 62 is identical to region 42. However, in other examples, the second directional locking region differs from region 42, such as by defining bristles, overlapping plates, or channels.

In some examples, the channels defined by the second directional locking region are complimentarily configured with ridges 46 of region 42. This enables ridges 46 to at least partially extend into the channels in an interlocking fashion. A suture disposed between ridges 46 and the channels is partially pressed into the channels by ridges 46. In this ridge-channel configuration, suture 28 may be more tightly retained by the cooperation of first and second clamp portions 40, 60.

In the example shown in FIG. 4, clip portion 26 may be selectively pressed against clip portion 27 by translating the two portions together. In other examples, clip portion 26 is pivotally connected to clip portion 27 to enable clip portion 26 to be selectively pressed against clip portion 27 by pivoting the two portions together. Any suitable type of hinge known in the art may be used, such as a flush, pivot, friction, surface, “T,” flag joint, strap, or butt hinge. Additionally or alternatively to a hinge, a flexible material or a binding may join first and second clip portions 26, 27 together in a manner that allows them to pivot relative to one another.

In the example shown in FIGS. 4, 7 and 8-11, clip portion 26 includes first posts 82 extending from support face 34. Posts 82 enable a user to conveniently manipulate clip portion 26, such as with a laparoscopic manipulation tool, tweezers, or with a user's fingers. Posts 82 may have any manner of shape and size effective for manipulating clip portion 26 and/or for interfacing with a particular tool. In the examples shown in FIGS. 4 and 7, clip portion 27 includes second posts 83 that are identical to first posts 82. In other examples, clip portion 27 does not include posts or includes posts that are different than posts 82.

First and second clip portions 26 and 27 collectively define a locking mechanism 70. In the example shown in FIG. 4, two locking mechanisms 70 are shown, one on the left side of the page and one on the right. In FIG. 4, locking mechanism 70 on the left side of the page includes a latch 71 defined by clip portion 26. Locking mechanism 70 also includes a catch 72 defined by clip portion 27. Locking mechanism 70 on the right side of FIG. 4 includes catch 72 defined by clip portion 26 and includes latch 71 defined by clip portion 27.

Locking mechanism 70 serves to selectively retain clip portion 26 in abutment with clip portion 27 when latch 71 selectively engages catch 72. In the example shown in FIG. 4, latch 71 forms a curved projection. As one skilled in the art of lock mechanisms will appreciate, latch 71 may take any variety of forms known in the art and function effectively as a latch. Catch 72 is complimentarily configured with latch 71 to enable catch 72 to receive and retain at least a portion of latch 71.

In the example shown in FIG. 4, latch 71 includes a weakened portion or notch 73 to enable a doctor to selectively cut off the end portion of latch 71. However, in the examples shown in FIGS. 8-11, first clip portion does not include a weakened portion. When latch 71 is severed, locking mechanism 70 no longer retains clip portion 26 in abutment with clip portion 27, and thus, no longer restricts suture 28 from moving. Weakened portion 73 may be an absence of material, a material relatively easy to cut, or a perforation.

With reference to FIG. 18, another example of clip 22 is described. In the example of clip 22 shown in FIG. 18, base member 30 and base member 50 are integrally connected and define a base slot 86 that extends to first and second clamp portions 40, 60. Base slot 86 enables a suture to be delivered to first and second clamp portions 40, 60 in a position between them.

In FIG. 18, clamp portion 60 is pivotally connected to clamp portion 40 by any suitable type of hinge, such as those described above. Clamp portion 60 may be selectively pivoted between an open position (shown in solid lines in FIG. 18) and a closed position (shown in dashed lines in FIG. 18). In the closed position, directional locking region 62 secures suture 28 between first and second clamp portions 40, 60. In other examples, clamp portion 60 is moveably mounted to clamp portion 40, such as on rails that enable clamp portion 60 to translate relative to clamp portion 40.

In the FIG. 18 example, first and second clamp portions 40, 60 collectively define a cutting slot 87 extending transverse to imaginary line 90. Cutting slot 87 provides access for a scalpel, knife, scissors or other cutting tool to cut a suture secured by clip 22 between first and second clamp portions 40, 60. Preferably, though not necessarily, cutting slot 87 is positioned proximate support faces 33, 53 to more readily divide the suture into a retained portion that is acted upon by first and second clamp portions 40, 60 and an unretained portion that is retained only weakly by first and second clamp portions 40, 60, or not retained at all.

In FIG. 18, a locking mechanism 74 is defined by first and second clamp portions 40, 60. Clamp portion 60 defines or includes a latch 75 with a curved end and clamp portion 40 defines a catch 76 with a curved recess. Latch 75 and catch 76 are complimentarily configured such that the curved end of latch 75 may mate with the curved recess of catch 76 to selectively secure clamp portion 60 to clamp portion 40.

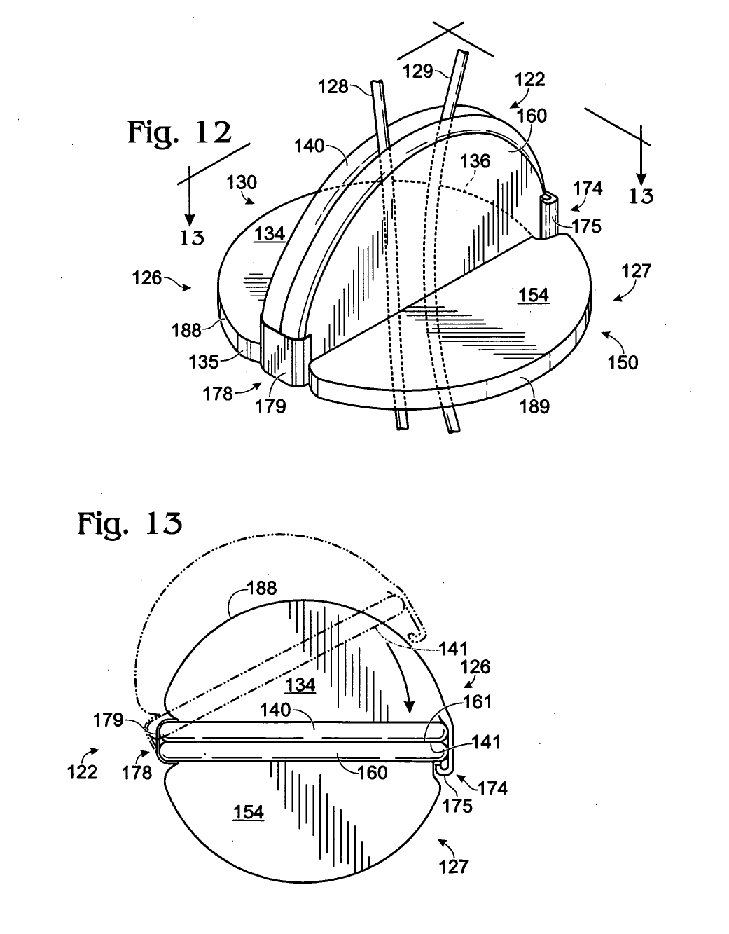

Turning attention to FIG. 12, second suture clip 122 is more fully described. Clip 122 includes a first clip portion 126 and a second clip portion 127 that are cooperatively configured to secure first suture 128 and second suture 129 between them. In other examples, clip 122 secures more than two sutures or secures a single suture. In the example shown in FIG. 12, clip portion 126 is identical to clip portion 127. However, in other examples, clip portion 126 is structurally different than clip portion 127.

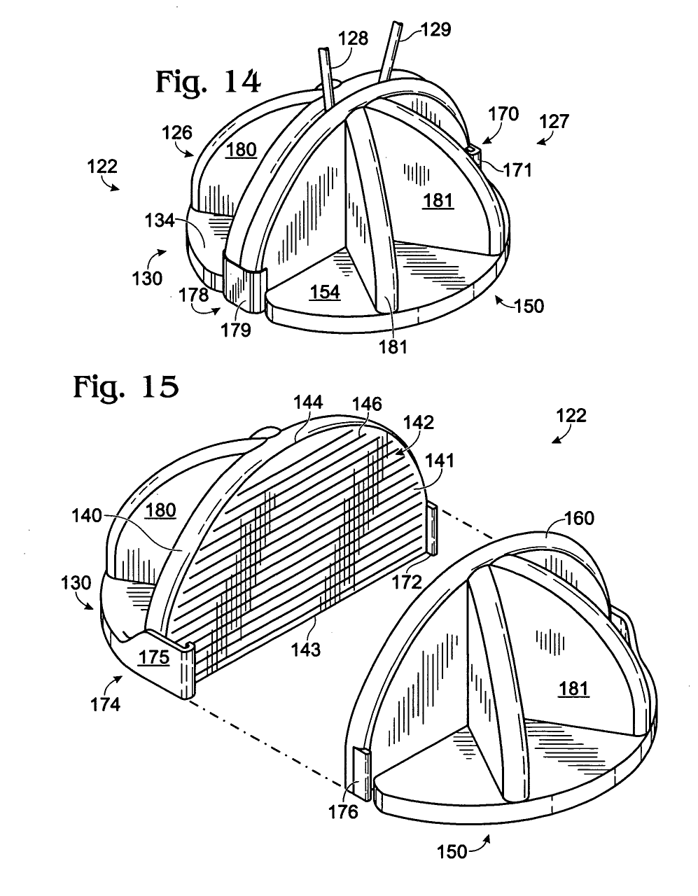

Clip portion 126 includes a first base member 130 and a first clamp portion 140. Clip portion 127 includes a second base member 150 and a second clamp portion 160. For ease of manufacturing, first and second clip portions 126, 127 are substantially similar to each other in the examples shown in FIGS. 12-17. In other examples, first and second clamp portions, 126, 127 include different features and designs. For the sake of brevity, clip portion 126 will be described below with the understanding that the description applies equally to clip portion 127, unless noted otherwise.

As shown in FIGS. 12-17, in similar fashion to base member 30, base member 130 includes a first pressure face 131, a first support face 134 and a first rear boundary surface 188 extending between a first shoulder 135 and a second shoulder 136. Base member 130 is substantially similar to base member 30 and thus need not be described again in detail. The same similarity exists between a second base member 150 and second base member 50 described above. Second base member 150 includes a second pressure face 151, a second support face 154 and a second rear boundary surface 189.

However, it is worth noting that the examples of first and second base members 130, 150 shown in FIGS. 12-17 do not include distinct abutment surfaces. Instead, as shown most clearly in FIG. 15, clamp portion 140 covers base member 130. Thus a first clamp face 141 of clamp portion 140 abuts with a corresponding clamp face of a second clamp portion 160.

In the examples shown in FIGS. 12-17, clamp portion 140 extends across base member 130 from shoulder 135 to shoulder 136. As shown in FIG. 15, clamp portion 140 defines a clamp face 141 that has a relatively broad surface area. Further, clamp face 141 defines a first directional locking region 142 with a relatively broad surface area.

The relatively broad surface area of first and second clamp portions 140, 160 enables them to secure sutures between them at almost any position between first and second shoulders 135, 136. Because sutures can be secured in a number of different positions, users of clip 122 need not concern themselves with precisely aligning sutures within a given space between first and second shoulders 135, 136. Moreover, the ample surface area of the clamp faces allows multiple sutures to be secured by first and second clamp portions 140, 160, such as first and second sutures 128, 129 shown in FIGS. 12 and 14.

In the example shown in FIG. 15, region 142 of clamp face 141 includes a plurality of ridges 146. Ridges 146 may be substantially similar to the ridges described above with regard to ridges 46, and accordingly will not be described again in detail. Further, ridges 146 may be arranged in any number of ridge patterns, such as those described above. The reader should understand that ridges 146 may be tilted toward a second end 144 of region 142 to restrict a suture from moving toward first end 143 while allowing the suture to move towards second end 144.

In other examples, region 142 includes additional or alternative features to restrict a suture from moving in a particular direction. Other features may include bristles, overlapping plates, or channels.

The examples of clips 122 shown in FIGS. 14-16 include first buttresses 180 supporting clamp portion 140 from base member 130 and include second buttresses 181 supporting clamp portion 160 from base member 140. First and second buttresses 180, 181 provide increased structural rigidity to first and second clip portions 126, 127 as well as provide convenient structures for manipulating first and second clip portions 126, 127. In some examples, buttresses 180, 181 are complimentarily configured with a clip delivery device (not pictured) to enable the clip delivery device to deliver a given clip from a magazine or cartridge of clips upon a user's input to the clip delivery device.

In the example shown in FIG. 17, clamp portion 140 is oriented at an oblique angle 159 relative to base member 130. Oblique angle 159 of clamp portion 140 causes second end 144 to extend closer toward clip portion 160 than first end 143 when clamp face 141 is facing second clip portion 160. As can be seen in FIG. 17, clamp portion 160 forms an oblique angle 159′ with base member 150.

In the FIG. 17 example of clip 122, clamp portion 140 pivots between an angled position (shown in solid lines) and an upright position where angle 159 is approximately 90°. In the upright position, clamp portion 140 contacts and is supported by buttresses 180. In some examples, clamp portion 140 pivots from the angled position to the upright position when acted on by clamp portion 160 as clamp portion 160 is pressed against clamp portion 140. The tilt of clamp portion 140 and/or of clamp portion 160 along with the pivoting interaction between the two clamp portions tends to increase the compressive force of the clamp portions 140, 160 on a suture disposed between them.

FIGS. 12-14 show examples of clip 122 that include a pivot mechanism 178 to enable clip portion 126 to pivot relative to clip portion 127. Pivot mechanism 178 includes a hinge 179, which in the example shown in FIGS. 12-14 is a flexible binding. In other examples, the hinge may be or include any of the hinges described above with regard to hinge 79.

In the example shown in FIG. 15, clip portion 126 is pressed against clip portion 127 by translating clip portion 126 toward clip portion 127. In FIG. 15, clip portion 126 is unattached to clip portion 127, but in some examples clip portion 126 is attached to clip portion 127 by rails (not pictured), which enable clip portions 126, 127 to translate relative to one another to a defined extent.

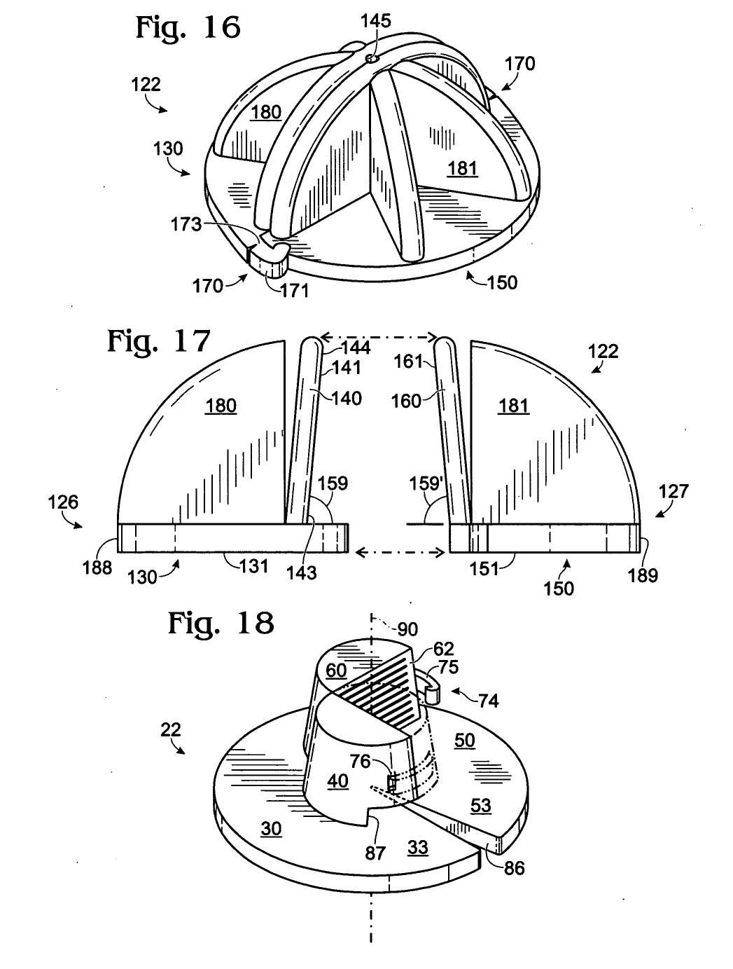

With reference to FIG. 16, a locking mechanism 170 substantially similar to locking mechanism 70 is briefly described. Locking mechanism 170 on the near side of FIG. 16 includes a latch 171 defined by base member 130 that mates with a catch defined in base member 150. Another locking mechanism 170 on the opposite side of clip 122 is also provided, but with the latch being defined by base member 150 and the catch being defined in base member 130. A weakened portion 173 of latch 171 enables latch 171 to be more easily cut to unlock clip 122.

An alternative locking mechanism 174 is shown in FIGS. 12-15. Locking mechanism 174 on the near side of FIG. 15 includes a latch 175 defined by clip portion 126 and a catch 176 defined by or coupled to clamp portion 160. On the far side of FIG. 15, another locking mechanism 174 is provided where the latch is defined by clip portion 127 and catch 176 is defined by or coupled to clamp portion 140. As can be seen in FIG. 15, latch 175 forms a hook that is complimentarily configured with catch 176.

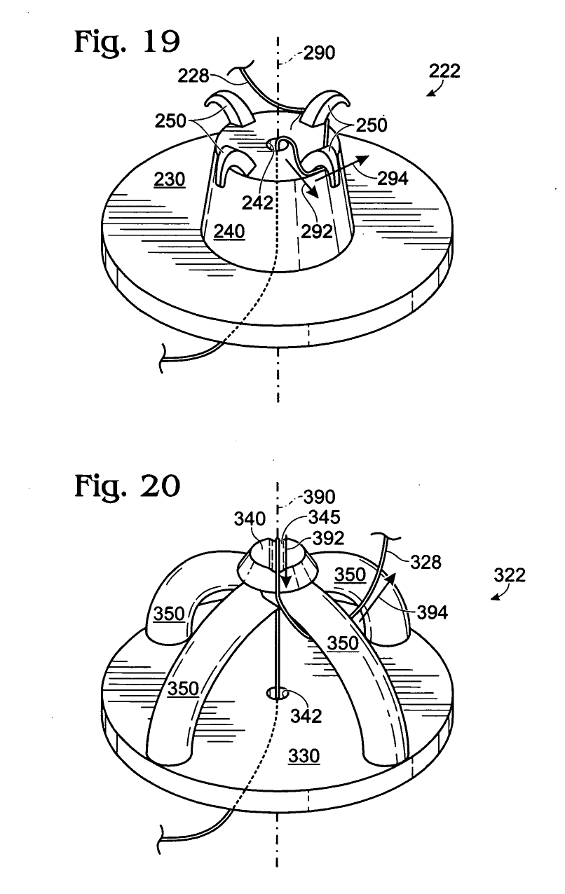

Turning attention to FIG. 19, a third suture clip 222 is described. Clip 222 includes a base member 230, a first securement member 240, and second securement members 250. Base member 230 is substantially similar to base members 30 and 130 described previously. As can be seen in FIG. 19, the bottom of base member 230, which may include any of the features discussed above with regard to pressure face 31, has an area that is significantly larger than the corresponding area of first securement member 240.

In the FIG. 19 example, first securement mechanism 240 is a bollard. Bollard 240 is similar to first and second clamp portions 40, 60 described above when those clamp portions are pressed together. However, in FIG. 19, bollard 240 is an integrally formed member instead of existing in separate first and second clamp portions. Bollard 240 defines a port 242 through which a suture 228 may pass through. It should be noted that port 242 continues through base member 230, that is, base member 230 defines a port in communication with port 242.

Port 242 may be cylindrical as shown in FIG. 19, or may be any variety of shapes, such as conical or tubular with rectangular, square, triangular, or oval cross sections. A characteristic dimension extending across port 242, such as the diameter of port 242 in FIG. 19, may be larger or smaller than that shown in FIG. 19.

For example, port 242 may have a diameter or characteristic dimension only slightly smaller than the diameter or characteristic dimension of bollard 240. Further, the diameter or characteristic dimension from one end of bollard 240 to an opposite end of bollard 240 may also be larger or smaller than shown in FIG. 19. In some examples, port 242 has a relatively small characteristic dimension, at least at the bottom of base member 230, to reduce incidence of tissue bulging into port 242, also known as herniating, when base member 230 exerts pressure onto the tissue.

In the example shown in FIG. 19, second securement members 250 are cleats or hooks projecting from the top of bollard 240. Cleats 250 provide securement points for suture 228. Looping suture 228 around one or more of cleats 250 inhibits suture 228 from being moved relative to bollard 240. In other examples, more and fewer cleats are provided. Further, the shape of cleats 250 may be altered to be any structure suitable for interacting with a suture to restrict the suture from moving, such as posts, rings, t-shaped members, v-shaped members or clips.

First and second securement members 240, 250 are configured to cooperate to secure suture 228, that is, to inhibit suture 228 from moving relative to clip 222. First and second securement members 240, 250 cooperate to guide suture 228 in a first direction 292 transverse to a port axis 290 extending within port 242 through clip 222. Second securement member 250 is further configured to guide suture 228 in a second direction 294 transverse to both port axis 290 and first direction 292 when suture 228 engages second securement member 250, such as by looping around it. Suture 228 is most readily redirected transverse to port axis 290 when it engages first and second securement members 240, 250 under tension.

With reference to FIG. 20, a fourth suture clip 322 is described. Clip 322 includes a base member 330, a first securement member 340, and second securement members 350. Clip 322 secures a suture 328 when suture 328 is looped around securement member 340 and one or more of securement members 350.

Base member 330 is substantially similar to base members 30, 130, and 230 described previously. However, as with base member 230, base member 330 defines a port 342 through which suture 328 may extend through. As can be seen in FIG. 20, the bottom of base member 330, which may include any of the features discussed above with regard to pressure face 31, has an area that is significantly larger than the cross sectional area of suture 328.

In the example shown in FIG. 20, first securement member 340 forms a cap, which is supported on second securement members 350 and defines a guide or channel 345 in which suture 328 may lie. As can be seen in FIG. 20, suture 328 may pass through port 342 and loop over cap 340. Suture 328 naturally settles into channel 345 because of the angled faces defining channel 345.

Second securement members 30 shown in FIG. 20 are arms or elongate arches. Arms 350 provide securement points for suture 328. Looping suture 328 around one or more of arms 350, alone or in combination with cap 350, inhibits suture 328 from being moved relative to clip 322. In other examples, more and fewer arms 350 are provided. Further, the shape of arms 350 may be altered to any structure suitable for interacting with a suture to restrict the suture from moving, such as posts, rings, t-shaped members, v-shaped members or pivoting clips.

First and second securement members 340, 350 are configured to cooperate to secure suture 328, that is, to inhibit suture 328 from moving relative to clip 322. First and second securement members 340, 350 cooperate to guide suture 328 in a first direction 392 transverse to a port axis 390 extending within port 342 through clip 322. Second securement member 350 is further configured to guide suture 328 in a second direction 394 transverse to both port axis 390 and first direction 392 when suture 328 engages second securement member 350, such as by looping around it. Suture 328 is most readily redirected transverse to port axis 390 when it engages first and second securement members 340, 350 under tension.

The suture clips described above may be formed from any material suitable for use during surgery. Suitable materials, such as bioinert materials, are well known in the art. In some examples, the suture clip is formed from a dissolvable material selected to dissolve away after its suture securing functionality is not longer required. Any known dissolvable material for use in surgical applications, as well as any known dissolvable material that is determined to be suitable for surgical applications, may be used to form the suture clips described above.

It is believed that the disclosure set forth above encompasses multiple distinct inventions with independent utility. While each of these inventions has been disclosed in a particular form, the specific embodiments thereof as disclosed and illustrated herein are not to be considered in a limiting sense as numerous variations are possible. The subject matter of the inventions includes all novel and non-obvious combinations and subcombinations of the various elements, features, functions and/or properties disclosed herein. Where the disclosure or subsequently filed claims recite “a” or “a first” element or the equivalent thereof, it is within the scope of the present inventions that such disclosure or claims may be understood to include incorporation of one or more such elements, neither requiring nor excluding two or more such elements.

Applicant reserves the right to submit claims directed to certain combinations and subcombinations that are directed to one or more of the disclosed inventions and that are believed to be novel and non-obvious. Inventions embodied in other combinations and subcombinations of features, functions, elements and/or properties may be claimed through amendment of those claims or presentation of new claims in the present application or in a related application. Such amended or new claims, whether they are directed to a different invention or directed to the same invention, whether different, broader, narrower or equal in scope to the original claims, are also regarded as included within the subject matter of the inventions of the present disclosure.

1. A suture clip, comprising:
a first clip portion and a second clip portion complimentarily configured with the first clip portion for securing a suture between the first and second clip portions when the first and second clip portions are selectively pressed together;
wherein the first clip portion comprises:
a first base member including a first pressure face and a first support face opposite the first pressure face, and
a first clamp portion extending from the first support face, the first clamp portion including a first clamp face defining a directional locking region having a first end proximate the first base member and a second end opposite the first end, the directional locking region being configured to permit the suture to move from the first end to the second end and to restrict the suture moving from the second end to the first end; and
wherein the second clip portion comprises:
a second base member defining a second pressure face and a second support face opposite the second pressure face, and
a second clamp portion extending from the second support face in a position aligned with the first clamp portion when the first and second clip portions are selectively pressed together, the second clamp portion including a second clamp face.

2. The suture clip of claim 1, wherein the first clip portion is pivotally connected to the second clip portion.

3. The suture clip of claim 1, wherein the second clamp face defines a second directional locking region.

4. The suture clip of claim 1, wherein the first clamp portion defines a cutting slot through which a portion of a cutting tool may be inserted to cut the suture secured by the first and second clip portions, the cutting slot being transverse to an imaginary line extending between the first end and the second end of the directional locking region.

5. The suture clip of claim 1, wherein:
the first base member includes a first shoulder and a second shoulder opposite the first shoulder; and
the first clamp portion is positioned between and spaced from both the first shoulder and the second shoulder.

6. The suture clip of claim 1, wherein:
the first base member includes a first shoulder and a second shoulder opposite the first shoulder; and
the first clamp portion extends across the first base member from the first shoulder to the second shoulder.

7. The suture clip of claim 6, wherein the first clip portion includes a buttress supporting the first clamp portion from the first base member.

8. The suture clip of claim 1, wherein:
the first base member includes a first abutment surface extending between the first pressure face and the first support face; and
the second base member includes a second abutment surface extending between the second pressure face and the second support face, the second abutment surface being complimentarily configured with the first abutment surface for positioning the first base member into flush abutment with the second base member when the first clip portion and the second clip portion are selectively pressed together.

9. The suture clip of claim 8, wherein the first abutment surface defines a projection and the second abutment surface defines a recess complimentarily configured with the projection.

10. The suture clip of claim 1, wherein the directional locking region defines a plurality of ridges that extend across the directional locking region transverse to a first imaginary line extending between the first end and the second end.

11. The suture clip of claim 10, wherein the ridges are tilted relative to the first clamp face toward the second end.

12. The suture clip of claim 10, wherein the ridges are arranged in substantially parallel rows oriented substantially perpendicular to the first imaginary line.

13. The suture clip of claim 10, wherein:
the first imaginary line divides the directional locking region into a first portion and a second portion;
the ridges extending in the first portion of the directional locking region are arranged in substantially parallel rows oriented at an oblique angle relative to the first imaginary line; and
the second portion forms a mirror image of the first portion.

14. The suture clip of claim 10, wherein:
the first imaginary line divides the directional locking region into a first portion and a second portion;
a second imaginary line transverse to the first imaginary line divides the first portion into a subpart A and into a subpart B and divides the second portion into a subpart C and into a subpart D;
the ridges extending in the subpart A are arranged into substantially parallel rows oriented substantially parallel to the second imaginary line;
the ridges extending in the subpart B are arranged in substantially parallel rows oriented at an oblique angle relative to the first imaginary line;
the subpart C of the directional locking region forms a mirror image of the subpart A of the directional locking region; and
the subpart D of the directional locking region forms a mirror image of the subpart B of the directional locking region.

15. The suture clip of claim 1, wherein the directional locking region defines a plurality of channels that extend across the directional locking region transverse to a first imaginary line extending between the first end and the second end.

16. The suture clip of claim 1, wherein the directional locking region defines a plurality of overlapping plates, the plates overlapping in a pattern wherein a portion of a given plate proximate the second end is exposed and a portion of the given plate distal the second end is covered by an adjacent plate in the pattern.

17. The suture clip of claim 1, wherein the directional locking region defines a plurality of bristles tilted relative to the first clamp face toward the second end.

18. The suture clip of claim 1, wherein the first pressure face is textured to increase its coefficient of friction to resist the first clip portion sliding relative to an organ when the first pressure face is placed in contact with the organ.

19. The suture clip of claim 1, wherein the first pressure face is coated with a hemostat material.

20. The suture clip of claim 1, wherein the first pressure face and the second pressure face have a combined area of at least 75 square millimeters in which to distribute pressure over an organ when the first clip portion and the second clip portion are selectively pressed together.

21. The suture clip of claim 1, further comprising a locking mechanism to selectively lock the first clip portion in a position abutting the second clip portion, the locking mechanism including:
a latch mounted to the first clip portion; and
a catch mounted to the second clip portion, the catch being complimentarily configured with the latch for selectively securing the latch to the catch.

22. The suture clip of claim 21, wherein the latch includes a weakened portion configured to allow the latch to be selectively severed to unlock the first clip portion from a position abutting the second clip portion.

23. The suture clip of claim 1, wherein:
the first clamp portion is oriented at an oblique angle relative to the first base member, the oblique angle of the first clamp portion causing the second end to extend closer toward the second clip portion than the first end when the first clamp face is facing the second clip portion; and
the first clamp portion is configured to pivot relative to the first base member when the first clamp portion is selectively pressed against the second clamp portion.

24. A suture clip, comprising:
a base member in the form of a planar disk defining a port through which a suture may pass, the port extending in a port axis; and
a retention assembly mounted on the base member, the retention assembly comprising:
a first securement member; and
a second securement member that is configured to cooperate with the first securement member to guide the suture in a first direction transverse to the port axis and in a second direction transverse to both the port axis and the first direction when the suture engages both the first securement member and the second securement member under tension.

25. The suture clip of claim 24, wherein:
the first securement member comprises a bollard defining a second port through which a suture may pass, the second port being in communication with the first port; and
the second securement member comprises a cleat coupled to the bollard.

26. The suture clip of claim 24, wherein:
the first securement member comprises a cap defining a groove; and
the second securement member comprises an arm supporting the cap.

27. A suture clip comprising:
a base member in the form of a planar disk defining an access slot into which a suture may be inserted;
a first clamp portion including a first clamp face, the first clamp portion being fixedly mounted to the base member adjacent the access slot such that the first clamp face is substantially inline with the access slot;
a second clamp portion moveably mounted to the first clamp portion, the second clamp portion moving between:
an open position where the second clamp portion is spaced from the first clamp portion and a suture can be inserted into the access slot and between the first and second clamp faces; and
a closed position where the second clamp face abuts the first clamp face to secure the suture between the first and second clamp faces.
